(19) United States
(12) Patent Application Publication
(10) Pub. No.: US 2025/0195341 A1
(43) Pub. Date: Jun. 19, 2025

(54) Personal Care Compositions Incorporating Natural Benefit Agents And Methods For The Same

(71) Applicant: Colgate-Palmolive Company, New York, NY (US)

(72) Inventors: David OLVERA, Col. Granada (MX); Sara MORALES, Mexico City (MX); Reina ESPINOSA, Atizapan de Zaragoza (MX); Luis Miguel LOPEZ, Atizapan de Zaragoza (MX); Antonia de la Cruz RAMIREZ MENDEZ, Mexico City (MX); Arisai GONZALEZ, Mexico City (MX); Qiang WU, Hillsborough, NJ (US)

(73) Assignee: Colgate-Palmolive Company, New York, NY (US)

(21) Appl. No.: 19/068,649

(22) Filed: Mar. 3, 2025

Related U.S. Application Data
(63) Continuation of application No. 17/871,111, filed on Jul. 22, 2022.
(60) Provisional application No. 63/225,202, filed on Jul. 23, 2021.

(51) Int. Cl. A61K 8/02; A61K 8/34; A61K 8/36; A61K 8/92; A61Q 15/00; A61Q 19/10; C11D 9/26; C11D 17/00

(52) U.S. Cl. CPC A61K 8/0216; A61K 8/345; A61K 8/361; A61K 8/922; A61Q 15/00; A61Q 19/10; C11D 9/265; C11D 17/0047; A61K 2800/30

(57) ABSTRACT

Solid cleansing compositions and methods for the same are disclosed herein. The solid cleansing composition may include a soap and a plant oil. The plant oil may include flaxseed oil, and may be present in an amount effective to maintain or increase hydration of skin, inhibit inflammation of skin, deodorize skin, inhibit irritation of skin, or combinations thereof.

### CROSS-REFERENCE TO RELATED APPLICATIONS

This application is a continuation of application Ser. No. 17/871,111 filed on Jul. 22, 2022 which claims the benefit of U.S. Provisional Patent Application No. 63/225,202, filed on Jul. 23, 2021, both of which are incorporated herein by reference to the extent consistent with the present disclosure.

### BACKGROUND

Conventional personal care products and compositions thereof often incorporate moisturizing agents to deliver or provide benefits to skin, thereby improving consumer perception of the personal care products. For example, moisturizing surfaces of skin is often a fundamental benefit of the moisturizing agents that improves consumer perception, as moisturizing surfaces of the skin improves skin feel. The most common moisturizing agents, however, are often synthetic or non-natural moisturizers and recent consumer perception regarding non-natural ingredients have motivated the development of more natural compositions or formulations via the inclusion of more naturally derived ingredients.

In view of the foregoing, efforts have been directed to reducing or eliminating non-natural ingredients from personal care products to provide improved consumer perception. For example, many manufacturers of personal care products are now providing or marketing more natural shampoos, conditioners, soaps, or other products to consumers. These more natural products, however, do not deliver moisturizing benefits comparable to the compositions incorporating the synthetic counterparts and many consumers do not want to compromise the moisturizing benefits of the non-natural ingredients for more natural ingredients.

What is needed, then, are improved personal care compositions incorporating natural moisturizing agents and having moisturizing benefits comparable to conventional personal care compositions incorporating non-natural moisturizing agents.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a solid cleansing composition including a soap and a plant oil. The plant oil may be present in an effective amount to maintain or increase hydration of skin, inhibit inflammation of skin, or inhibit irritation of skin.

A further aspect of the present disclosure is providing a method of ameliorating a negative effect of aloe on odor reduction of a cleansing composition. The method comprises:
(a) identifying a first odor reduction of aloe on the cleansing composition, comprising: (i) providing a first composition comprising a soap, wherein the first composition has a first odor reduction percentage; and (ii) adding aloe to the first composition to produce a second composition having a second odor reduction percentage; wherein the difference (or the normalized difference) between the first odor reduction percentage and the second odor reduction percentage is the first odor reduction of aloe on the cleansing composition;
(b) identifying a second odor reduction of aloe on the cleansing composition, comprising: (iii) providing a third composition comprising a plant oil, a hydrolyzed protein, and the first composition, wherein the third composition has a third odor reduction percentage; (iv) adding aloe to the third composition, to produce a fourth composition having a fourth odor reduction percentage; wherein the difference (or the normalized difference) between the third odor reduction percentage and the fourth odor reduction percentage is the second odor reduction of aloe on the cleansing composition; and
(c) applying the fourth composition to a skin for reducing odor of the skin. The second odor reduction of aloe on the cleansing composition is less than the first odor reduction of aloe on the cleansing composition, indicating the negative effect of aloe is ameliorated in the presence of the plant oil and the hydrolyzed protein.

In at least one implementation, the soap may include C₁₆-C₁₈ fatty acids and C₁₂-C₁₄ fatty acids. The C₁₆-C₁₈ fatty acids may be present in an amount of from about 60 weight % to about 80 weight %, based on the total weight of the solid cleansing composition. The C₁₂-C₁₄ fatty acids may be present in an amount of from about 20 weight % to about 40 weight %, based on the total weight of the solid cleansing composition.

In at least one implementation, the plant oil may include flaxseed oil. The plant oil may be present in an amount of from greater than 0 weight % to less than or equal to 5 weight %, optionally, in an amount of from greater than 0 weight % to less than or equal to 1 weight %, further optionally, in an amount of about 0.5 weight %, based on the total weight of the solid cleansing composition.

In at least one implementation, the solid cleansing composition may be free of polyquaternium-6.

In at least one implementation, the solid cleansing composition may be free of petrolatum.

In at least one implementation, the solid cleansing composition may include lather and skin feel attributes comparable to or relatively greater than a solid cleansing comprising polyquaternium-6, petrolatum, or combinations thereof.

In at least one implementation, the soap may include sodium soap, optionally the soap may include one or more of an ammonium soap, a potassium soap, a magnesium soap, a calcium soap, or combinations thereof.

In at least one implementation, the soap may include alkali metal salts of aliphatic acids having 8 to 22 carbon atoms.

In at least one implementation, the solid cleansing composition further includes one or more humectants, optionally, the one or more humectants may be selected from ascorbic acid, ascorbyl dipalmitate, acetamide MEA, glucose glutamate, glucuronic acid, TEA-lactate, TEA-PCA, corn syrup, fructose, glucose, glycerin, glycol, 1,2,6-hexanetriol, sodium lactate, sodium PCA, hydrogenated starch hydrolysate, inositol, lactic acid, lactose, mannitol, PCA, PEG-10 propylene glycol, polyamino sugar condensate, propylene glycol, pyridoxine dilaurate, saccharide hydrolysate, hydroxystearyl methylglucamine, glucamine, maltitol, mannitol, methyl gluceth-10, methyl gluceth-20, riboflavin, PEG-4, PEG-6, PEG-8, PEG-9, PEG-10, PEG-12, PEG-14, PEG-16, PEG-18, PEG-20, PEG-32, PEG-40, glutamic acid, glycereth-7, glycereth-12, glycereth-26, saccharide isomerate, sorbeth-20, sorbitol, sucrose, thioglycerin, tris-(hydroxymethyl)nitromethane, tromethamine, histidine, PEG-75, PEG-135, PEG-150, PEG-200, PEG-5 pentaerythritol ether, polyglyceryl sorbitol, sorbitol, urea, xylitol, or combinations thereof.

In at least one implementation, the humectants include glycerin.

In at least one implementation, the solid cleansing composition further includes one or more free fatty acids. The one or more free fatty acids may include one or more of palm kernel oil, palm oil, coconut oil, olive oil, laurel oil, or combinations thereof.

In at least one implementation, the solid cleansing composition may be a bar soap.

In at least one implementation, the solid cleansing composition may include lather and skin feel attributes comparable to or relatively greater than a solid cleansing including polyquaternium-6, petrolatum, or combinations thereof.

In at least one implementation, the solid cleansing composition includes a natural origin index of greater than 80%.

In at least one implementation, the solid cleansing composition includes a natural origin index of greater than 90%.

In at least one implementation, the solid cleansing composition includes a natural origin index of greater than 95%.

In at least one implementation, the first odor reduction of aloe on the cleansing composition is X=M1−M2 (or normalized X₀=(M1−M2)/M1), in which M1 stands for the first odor reduction percentage and M2 stands for the second odor reduction percentage. The second odor reduction of aloe on the cleansing composition is Y=M3−M4 (or normalized Y₀=(M3−M4)/M3), in which M3 stands for the third odor reduction percentage and M4 stands for the fourth odor reduction percentage. Y<X and Y₀<X₀.

The foregoing and/or other aspects and utilities embodied in the present disclosure may be achieved by providing a method for maintaining or increasing hydration of skin of a user. The method may include contacting any of the solid cleansing compositions disclosed herein with the skin of the user in need thereof.

The foregoing and/or other aspects and utilities embodied in the present disclosure may be achieved by providing a use of any of the solid cleansing compositions disclosed herein for maintaining or increasing hydration of skin.

The foregoing and/or other aspects and utilities embodied in the present disclosure may be achieved by providing a method for decreasing inflammation or irritation of skin of a user in need thereof. The method may include contacting any of the solid cleansing compositions disclosed herein with the skin of the user.

In at least one implementation, decreasing inflammation or irritation of skin may include producing natural moisturizing factors (NMFs) in the skin.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that one or more polycationic polymers or polyquats, such as Polyquaternium-6 (PQ-6), or a combination of PQ-6 and petrolatum in conventional personal care compositions may be replaced with flaxseed oil to thereby provide a more natural personal care composition having attributes (e.g., skin feel, lather, etc.) comparable or relatively greater than the conventional personal care compositions. For example, the present inventors have surprisingly and unexpectedly discovered that all the polyquats or a combination of the polyquats and petrolatum in conventional personal care compositions may be replaced with flaxseed oil to thereby provide a more natural personal care composition having attributes (e.g., skin feel, lather, etc.) comparable or relatively greater than the conventional personal care compositions.

Compositions disclosed herein may be or include solid cleansing compositions. Illustrative solid cleansing compositions may be or include, but are not limited to, bar soap compositions, cleansing bars, and other solid cleansing compositions that may be used for personal cleansing or as a laundry bar. The solid cleansing compositions may include one or more plant oils, one or more hydrolyzed proteins, or combinations thereof. Each of the one or more plant oils and/or the one or more hydrolyzed proteins may be present in an amount sufficient to provide attributes comparable to or greater than (e.g., significantly greater than) conventional personal care compositions excluding the one or more plant oils and/or the one or more hydrolyzed proteins. Each of the one or more plant oils and/or the one or more hydrolyzed proteins may also be present in an amount sufficient to provide attributes comparable to or greater than (e.g., significantly greater than) conventional personal care compositions incorporating a polyquat, such as PQ-6 or a combination of PQ-6 and petrolatum.

In a preferred implementation, the solid cleansing compositions disclosed herein may be free or substantially free of one or more polyquats or polyquaternium polymers, such as PQ-6, petrolatum, or combinations thereof. Polyquaternium or polyquat is the International Nomenclature for Cosmetic Ingredients (INCI) designation for several polycationic polymers that are used in the personal care industry. Polyquaternium is a neologism used to emphasize the presence of quaternary ammonium centers in the polymer. INCI has approved at least 37 different polymers under the polyquaternium designation. Different polymers are distinguished by the numerical value that follows the word “polyquaternium”. Polyquaternium-5, polyquaternium-7, and polyquaternium-47 are three examples, each a chemically different type of polymer. The numbers are assigned in the order in which they are registered rather than because of their chemical structure. Illustrative polyquaternium polymers may be or include, but are not limited to, Polyquaternium-1 (Ethanol, 2,2′,2″-nitrilotris-, polymer with 1,4-dichloro-2-butene and N,N,N′,N′-tetramethyl-2-butene-1,4-diamine), Polyquaternium-2 (Poly[bis(2-chloroethyl) ether-alt-1,3-bis[3-(dimethylamino)propyl]urea]), Polyquaternium-4 (Hydroxyethyl cellulose dimethyl diallylammonium chloride copolymer; Diallyldimethylammonium chloride-hydroxyethyl cellulose copolymer), Polyquaternium-5 (Copolymer of acrylamide and quaternized dimethylammoniumethyl methacrylate), Polyquaternium-6 (Poly(diallyldimethylammonium chloride)), Polyquaternium-7 (Copolymer of acrylamide and diallyldimethylammonium chloride), Polyquaternium-8 (Copolymer of methyl and stearyl dimethylaminoethyl ester of methacrylic acid, quaternized with dimethylsulphate), Polyquaternium-9 (Homopolymer of N,N-(dimethylamino) ethyl ester of methacrylic acid, quaternized with bromomethane), Polyquaternium-10 (Quaternized hydroxyethyl cellulose), Polyquaternium-11 (Copolymer of vinylpyrrolidone and quaternized dimethylaminoethyl methacrylate), Polyquaternium-12 (Ethyl methacrylate/abietyl methacrylate/diethylaminoethyl methacrylate copolymer quaternized with dimethyl sulfate), Polyquaternium-13 (Ethyl methacrylate/oleyl methacrylate/diethylaminoethyl methacrylate copolymer quaternized with dimethyl sulfate), Polyquaternium-14 (Trimethylaminoethylmethacrylate homopolymer), Polyquaternium-15 (Acrylamide-dimethylaminoethyl methacrylate methyl chloride copolymer), Polyquaternium-16 (Copolymer of vinylpyrrolidone and quaternized vinylimidazole), Polyquaternium-17 (Adipic acid, dimethylaminopropylamine and dichloroethylether copolymer), Polyquaternium-18 (Azelaic acid, dimethylaminopropylamine and dichloroethylether copolymer), Polyquaternium-19 (Copolymer of polyvinyl alcohol and 2,3-epoxypropylamine), Polyquaternium-20 (Copolymer of polyvinyl octadecyl ether and 2,3-epoxypropylamine), Polyquaternium-22 (Copolymer of acrylic acid and diallyldimethylammonium Chloride), Polyquaternium-24 (Quaternary ammonium salt of hydroxyethyl cellulose reacted with a lauryl dimethyl ammonium substituted epoxide), Polyquaternium-27 (Block copolymer of Polyquaternium-2 and Polyquaternium-17), Polyquaternium-28 (Copolymer of vinylpyrrolidone and methacrylamidopropyl trimethylammonium), Polyquaternium-29 (Chitosan modified with propylen oxide and quaternized with epichlorhydrin), Polyquaternium-30 (Ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quatemized with diethylsulfate bound to a block of polyacrylonitrile), Polyquaternium-32 (Poly (acrylamide 2-methacryloxyethyltrimethyl ammonium chloride)), Polyquaternium-33 (Copolymer of trimethylaminoethylacrylate salt and acrylamide), Polyquaternium-34 (Copolymer of 1,3-dibromopropane and N,N-diethyl-N′,N′-dimethyl-1,3-propanediamine), Polyquaternium-35 (Methosulphate of the copolymer of methacryloyloxyethyltrimethylammonium and of methacryloyloxyethyldimethylacetylammonium), Polyquaternium-36 (Copolymer of N,N-dimethylaminoethylmethacrylate and buthylmethacrylate, quaternized with dimethylsulphate), Polyquaternium-37 (Poly(2-methacryloxyethyltrimethylammonium chloride)), Polyquaternium-39 (Terpolymer of acrylic acid, acrylamide and diallyldimethylammonium Chloride),

Polyquaternium-42 (Poly[oxyethylene(dimethylimino)ethylene (dimethylimino)ethylene dichloride]), Polyquaternium-43 (Copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (Copolymer of (N-methyl-N-ethoxyglycine)methacrylate and N,N-dimethylaminoethylmethacrylate, quaternized with dimethyl sulphate), Polyquaternium-46 (Terpolymer of vinylcaprolactam, vinylpyrrolidone, and quaternized vinylimidazole), and Polyquaternium-47 (Terpolymer of acrylic acid, methacrylamidopropyl trimethylammonium chloride, and methyl acrylate). In an exemplary implementation, the solid cleansing compositions disclosed herein are free or substantially free of polyquaternium-6 (PQ-6), a polymeric quaternary ammonium salt derived from the homopolymerization of diallyldimethylammonium chloride (DADMAC) monomer.

The compositions disclosed herein may have a natural origin index of greater than about 80%. As used herein, the term or expression “natural origin index” may refer to a value (e.g., a percentage) or degree of a substance (e.g., a product or a composition thereof) that describes the natural content of ingredients or formulation of the substance. The natural origin index may be determined by the ISO 16128-2:2017 standard of the International Organization for Standardization (ISO). In at least one example, the natural origin index of the personal care compositions disclosed herein may be greater than 80%, greater than 85%, greater than 90%, greater than 95%, greater than 96%, greater than 97%, greater than 98%, greater than 99%, greater than 99.5%, or 100%.

The solid cleansing compositions disclosed herein may exhibit comparable, enhanced, improved, or relatively greater (e.g., statistically significantly greater) attributes, properties, and/or features as compared to solid cleansing compositions including a polyquaternium or polyquat, such as PQ-6, petrolatum, or any combination thereof. For example, the solid cleansing compositions disclosed herein may exhibit substantially similar or relatively greater performance with respect to lather, skin feel, or combinations thereof, as compared to a conventional personal care composition including any one or more of a polyquat, petrolatum, or any combination thereof. It should be appreciated that lather may refer to any one or more of the following: bar glide on hands, flash foam, bubble size, stickiness, foam amount, whiteness of foam, density of foam, peaking, foam thickness on hands, slipperiness of hands (wet), slipperiness of hands (dry), bar glide on arms, foam thickness on arm, time to rinse, or combinations thereof. It should further be appreciated that skin feel may refer to any one or more of the following: wet slip feel under water, amount of residue when wet, slipperiness after rinsing, tightness, tackiness, slipperiness, smoothness, gloss, white residue after washing, amount of residue after washing, or combinations thereof. As used herein, the term or expression “substantially similar” may refer to a property or value of a test composition that does not change (e.g., increase or decrease) from a comparative composition by an amount greater than 0.5%, greater than 1%, greater than 1.5%, greater than 2%, greater than 2.5%, greater than 3%, greater than 3.5%, greater than 4%, greater than 4.5%, greater than 5%, greater than 6%, greater than 7%, greater than 8%, greater than 9%, or greater than 10%.

As used herein, the term or expression “personal care composition” may refer to a composition for topical application to skin of mammals, especially humans. The personal care composition may generally be a solid cleansing, and may include any product applied to a human body. The personal care composition may be in any suitable form. Illustrative forms of the personal care composition may be or include, but is not limited to, a liquid, a lotion, a cream, a foam, a scrub, a gel, a soap bar, a toner, applied with an implement or via a face mask or a scalp mask, or the like. In a preferred implementation, the personal care composition is a bar soap.

As used herein, the term or expression “bar glide” may refer to a tactile attribute or property that evaluates the ease of movement or sliding of the bar soap across the skin. The bar glide may be evaluated by rubbing a bar soap on the skin (e.g., skin of arm) to evaluate the resistance between the bar soap and the skin. The bar glide may be determined on a scale of 0 to 10.

As used herein, the term or expression “foam thickness” may refer to a tactile attribute or property that evaluates a force required or necessary to press the foam. The foam thickness may be evaluated by pressing a dry finger on foam after formation. The foam may be generated at three different sites, such as three difference sites along an arm from the elbow to the wrist. The foam thickness may be determined on a scale of 0 to 10.

As used herein, the term or expression “rinse time” may refer to a time necessary to rinse a soap feeling on skin. The rinse time may be evaluated by running one or more fingers along the skin during rinsing and determining the amount of time when the bar soap feel is completely gone.

As used herein, the term or expression “skin slipperiness” may refer to a tactile attribute that evaluates the ease of moving fingers across the skin. The skin slipperiness may be evaluated by moving a finger (e.g., index finger) across the skin in a single stroke. The skin slipperiness may be determined on a scale of 0 to 10.

As used herein, the term or expression “tightness” may refer to a tactile attribute that evaluates the presence of taut or drawn skin. The tightness may be evaluated by twisting a portion of the skin, such as the skin of an arm, and lifting the arm upward once to evaluate the feel. The tightness may be determined on a scale of 0 to 10.

As used herein, the term or expression “tackiness” may refer to a tactile attribute that evaluates the degree in which fingers adhere to skin. The tackiness may be evaluated by tapping one or more sites or places across the skin (e.g., skin of arm) using one or more fingers. The tackiness may be determined on a scale of 0 to 10.

As used herein, the term or expression “smoothness” may refer to a tactile attribute that evaluates the absence of roughness on the skin. The smoothness may be evaluated by moving a finger from the elbow to the wrist in a downward stroke one or more times without pressing. The smoothness may be determined on a scale of 0 to 10.

As used herein, the term or expression “gloss” may refer to a visual attribute that evaluates the amount of reflected light or sheen after washing skin. The gloss may be evaluated by holding the skin surface upward at a 45° angle against reflected light (e.g., holding arm upward at 45° angle). The gloss may be determined on a scale of 0 to 10.

As used herein, the term or expression “whiteness residue” may refer to a visual attribute that evaluates the amount of white or ashen skin. The whiteness residue may be evaluated by holding the skin surface downward at a 45° angle and observe the inner part of the arm from elbow to the wrist. The whiteness residue may be determined on a scale of 0 to 10.

As used herein, the term or expression “amount of residue” may refer to a tactile attribute that evaluates any deposition of residue on skin. The amount of residue may be evaluated by using a dry finger to collect any residue from the elbow to the wrist. The amount of residue may be determined on a scale of 0 to 10.

As used herein, the term or expression “type of residue” may refer to a characterization of the type of residue on the skin. The type of residue may be evaluated by moving a finger along the skin and determining one or more of the following qualitative properties: oily, greasy, waxy, powdery, slimy, or combinations thereof. A reference for oily is mineral oil, a reference for greasy is Vaseline, a reference for waxy is a candle, a reference for powdery is talc powder, and a reference for slimy is egg white.

As used herein, the term or expression “water droplets” may refer to a visual attribute that assesses the presence or absence of stable drops of water on the skin. The water droplets may be evaluated by wetting skin, such as skin of an arm, under water one or more times and evaluating the presence or absence of water droplets.

The solid cleansing composition may include one or more plant oils. As used herein, “plant oil” may refer to oil that is obtained from a plant, or manufactured oil made by blending at least two components of oil (e.g., triglycerides, saturated and/or unsaturated fatty acids, etc.) to substantially mimic the composition of a natural plant oil or provide an oil substantially similar in composition to a plant oil. For example, a manufactured oil substantially similar in composition to a plant oil may include at least 50 weight %, at least 60 weight %, at least 70 weight %, at least 80 weight %, at least 90 weight %, at least 95 weight %, at least 98 weight %, at least 99 weight %, at least 99.5 weight %, at least 99.9 weight %, or 100 weight % of the components that are naturally found in the plant oil that the manufactured oil is designed to substantially mimic.

Illustrative plant oils may be or include, but are not limited to, palm kernel oil, coconut oil, avocado oil, canola oil, corn oil, cottonseed oil, olive oil, palm oil, high-oleic sunflower oil, mid-oleic sunflower oil, sunflower oil, palm stearin oil, palm kernel olein oil, safflower oil, babassu oil, sweet almond oil, castor oil, canola oil, soybean oil, olive oil, acai oil, andiroba oil, apricot kernel oil, argan oil, passion fruit oil, marula oil, mango oil, shea oil, macadamia nut oil, brazil nut oil, borage oil, copaiba oil, grape seed oil, buriti oil, sesame oil, flaxseed oil or linseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, hemp oil, and the like, and mixtures or combinations thereof. In a preferred implementation, the plant oil includes flaxseed or linseed oil.

The amount or concentration of the one or more plant oils present in the solid cleansing composition may vary widely. In at least one implementation, the amount of the one or more plant oils present in the solid cleansing composition may be from greater than 0 weight % to less than or equal to 5 weight %, based on the total weight of the solid cleansing composition. For example, the amount of the one or more plant oils present in the solid cleansing composition may be from greater than 0 weight %, about 0.5 weight %, about 1 weight %, about 1.5 weight %, about 2 weight %, or about 2.5 weight % to about 3 weight %, about 3.5 weight %, about 4 weight %, about 4.5 weight %, or about 5 weight %, based on the total weight of the solid cleansing composition. In another example, the amount of the one or more plant oils present in the solid cleansing composition may be from greater than 0 weight % to about 5 weight %, about 0.5 weight % to about 4.5 weight %, about 1 weight % to about 4 weight %, about 1.5 weight % to about 3.5 weight %, or about 2 weight % to about 3 weight %. In another implementation, the amount of the one or more plant oils present in the solid cleansing composition may be from greater than 0 weight % to less than or equal to 1 weight %, based on a total weight of the solid cleansing composition. For example, the amount of the one or more plant oils present in the solid cleansing composition may be from greater than 0 weight %, about 0.1 weight %, about 0.2 weight %, about 0.3 weight %, about 0.4 weight %, or about 0.45 weight % to about 0.5 weight %, about 0.6 weight %, about 0.7 weight %, about 0.8 weight %, about 0.9 weight %, or about 1.0 weight %. In another example, the amount of the one or more plant oils present in the solid cleansing composition may be from greater than 0 weight % to about 1.0 weight %, about 0.1 weight % to about 0.9 weight %, about 0.2 weight % to about 0.8 weight %, about 0.3 weight % to about 0.7 weight %, about 0.4 weight % to about 0.6 weight %, or about 0.45 weight % to about 0.5 weight %.

The solid cleansing composition may include one or more hydrolyzed proteins. The hydrolyzed proteins may be completely hydrolyzed or partially hydrolyzed. Illustrative hydrolyzed proteins may be or include, but are not limited to, hydrolyzed gelatin, hydrolyzed collagen, hydrolyzed casein, hydrolyzed whey protein, hydrolyzed milk protein, hydrolyzed soy protein, hydrolyzed egg protein, hydrolyzed wheat protein, amino acids, peptides, and the like, or combinations thereof. In a preferred implementation the hydrolyzed protein includes hydrolyzed milk protein, such as CAS 92797-39-2 (EINECS: 296-575-2). For example, the hydrolyzed protein may be or include a hydrolyzed phosphoprotein derived from natural dairy protein, such as MILK TEIN NPNF®, which is commercially available from Tri-K Industries Inc. of Denville, NJ.

The hydrolyzed milk protein may be or include milk protein hydrolyzed by an enzyme. For example, a milk protein may be enzymatically hydrolyzed to provide the hydrolyzed milk protein.

The hydrolyzed protein may be a solution or mixture. In one implementation, the hydrolyzed protein may be provided as a pure or substantially pure solution. In another implementation, the hydrolyzed protein may be provided as a solution including one or more hydrolyzed proteins dissolved, mixed, or otherwise dispersed in the solution. In at least one implementation, the hydrolyzed protein may be an aqueous solution including the hydrolyzed protein in an amount of from greater than 0 weight % to less than or equal to 50 weight %. For example, a hydrolyzed protein solution may be an aqueous solution including one or more hydrolyzed proteins in an amount of from greater than 0 weight %, about 5 weight %, about 10 weight %, about 15 weight %, about 18 weight %, or about 20 weight % to about 25 weight %, about 30 weight %, about 35 weight %, about 40 weight %, about 45 weight %, or about 50 weight %. In another example, the hydrolyzed protein solution may be an aqueous solution including one or more hydrolyzed proteins in an amount of from greater than 0 weight % to about 50 weight %, about 5 weight % to about 45 weight %, about 10 weight % to about 40 weight %, about 15 weight % to about 35 weight %, about 20 weight % to about 30 weight %, or about 20 weight % to about 25 weight %. In a preferred implementation, the hydrolyzed protein solution is an aqueous solution including about 22 weight % of the hydrolyzed protein in water. In a more preferred implementation, the hydrolyzed protein solution is an aqueous solution including about 22 weight % of hydrolyzed milk protein in water.

The amount or concentration of the hydrolyzed protein solution present in the solid cleansing composition may vary widely. In at least one implementation, the amount of the hydrolyzed protein solution (e.g., a 22 weight % solution of hydrolyzed proteins) present in the solid cleansing composition may be from greater than 0 weight % to less than or equal to 10 weight %, based on the total weight of the solid cleansing composition. For example, the amount of the hydrolyzed protein solution present in the solid cleansing composition may be from greater than 0 weight %, about 1 weight %, about 2 weight %, about 3 weight %, about 4 weight %, or about 5 weight % to about 6 weight %, about 7 weight %, about 8 weight %, about 9 weight %, or about 10 weight %, based on a total weight of the solid cleansing composition. In another example, amount of the hydrolyzed protein solution present in the solid cleansing composition may be from greater than 0 weight %, about 0.2 weight %, about 0.4 weight %, about 0.6 weight %, about 0.8 weight % or about 1.0 weight % to about 1.2 weight %, about 1.4 weight %, about 1.6 weight %, about 1.8 weight %, or about 2.0 weight %, based on the total weight of the solid cleansing composition.

The amount or concentration of the one or more hydrolyzed proteins present in the solid cleansing composition may vary widely. In at least one implementation, the amount of the one or more hydrolyzed proteins present in the solid cleansing composition may be from greater than 0 weight % to less than or equal to 1 weight %, based on the total weight of the solid cleansing composition. For example, the amount of the one or more hydrolyzed proteins present in the solid cleansing composition may be from greater than 0 weight %, about 0.05 weight %, about 0.1 weight %, about 0.15 weight %, about 0.2 weight %, about 0.25 weight %, about 0.3 weight %, about 0.35 weight %, about 0.4 weight %, about 0.45 weight %, or about 0.5 weight % to about 0.55 weight %, about 0.6 weight %, about 0.65 weight %, about 0.7 weight %, about 0.75 weight %, about 0.8 weight %, about 0.85 weight %, about 0.9 weight %, about 0.95 weight %, or about 1 weight %, based on the total weight of the solid cleansing composition. In a preferred implementation, the amount of the one or more hydrolyzed proteins present in the solid cleansing composition may be from about 0.15 weight % to about 0.3 weight %, more preferably about 0.2 weight % to about 0.25 weight %, even more preferably about 0.22 weight %, based on the total weight of the solid cleansing composition.

The one or more plant oils and/or the one or more hydrolyzed proteins may each be present in an effective or a therapeutically effective amount. As used herein, the expression or term “effective amount” may refer to an amount of the plant oil and/or an amount of the hydrolyzed protein sufficient to elicit a synergistic effect or elicit a response (e.g., biological medical, etc.) of a tissue, system, animal, or human that is being sought. For example, the plant oil and/or the hydrolyzed protein may each be present in the solid cleansing composition in an effective amount to reduce inflammation and/or irritation of skin. In another example, the plant oil and/or the hydrolyzed protein may each be present in the solid cleansing composition in an effective amount to maintain or increase hydration of skin.

In an exemplary implementation, the solid cleansing composition excludes the one or more hydrolyzed proteins. For example, the solid cleansing composition may be free or substantially free of the hydrolyzed proteins. Accordingly, in an exemplary implementation, the solid cleansing composition may include flaxseed oil and exclude the hydrolyzed proteins.

The solid cleansing composition may include at least one cleansing component. In at least one implementation, the cleansing component may include a base component, such as a soap or a soap chip. The base component or the soap may be or include alkali metal or alkanol ammonium salts of aliphatic alkane- or alkene-monocarboxylic acids, including about 6 to about 22 carbon atoms, about 6 to about 18 carbon atoms, or about 12 to about 18 carbon atoms. Illustrative soaps that may be utilized in the solid cleansing composition may be or include, but are not limited to, sodium soaps, ammonium soaps, potassium soaps, magnesium soaps, calcium soaps, and the like, or mixtures thereof. In a preferred implementation, the base component or soap includes a sodium soap; however, it should be appreciated that at least a portion of the soap may also include one or more ammonium soaps, potassium soaps, magnesium soaps, calcium soaps, or the like, or mixtures and combinations thereof. In a preferred implementation, the base component or the soap may be or include, but is not limited to, alkali metal salts of aliphatic (alkanoic or alkenoic) acids having about 8 to about 22 carbon atoms or about 10 to about 20 carbon atoms.

The base component or soap may be a fatty acid soap. The fatty acid soap may include one or more neutralized fatty acids. Illustrative fatty acids used for the fatty acid soap may be or include, but are not limited to, myristic acid, lauric acid, palmitic acid, oleic acid, stearic acids, or the like, or combinations thereof. Sources of fatty acids may include coconut oil, palm oil, grape seed oil, palm kernel oil, tallow, avocado, canola, corn, cottonseed, olive, hi-oleic sunflower, mid-oleic sunflower, sunflower, palm stearin, palm kernel olein, safflower, babassu oils, or combinations thereof.

The fatty acids may be neutralized with any base to form the soap or fatty acid soap. Illustrative bases may be or include, but are not limited to, sodium hydroxide, potassium hydroxide, triethanolamine, or the like, or mixtures and combinations thereof. In certain implementations, the fatty acid soap may be formed from fatty acids neutralized by two or more bases. In certain embodiments, the bases are sodium hydroxide and triethanolamine. In certain implementations, the molar ratio of sodium hydroxide and triethanolamine is 1:1. In certain implementations, the fatty acids may be or include any one or more of oleic acid, palmitic acid, stearic acid, lauric acid, or combinations thereof. For example, the fatty acid soap may be or include sodium palmitate, sodium oleate, sodium laurate, sodium stearate, or any combination or mixture thereof. In at least one implementation, the fatty acid soap may further include glycerin.

The amount or concentration of the soap in the solid cleansing component may vary widely. In at least one implementation, the amount of the soap in the solid cleansing component may be greater than or equal to 50 weight % and less than or equal to 95 weight %. For example, the amount of the soap in the solid cleansing component may be from about 50 weight %, about 55 weight %, about 60 weight %, about 65 weight %, or about 70 weight % to about 75 weight %, about 80 weight %, about 85 weight %, about 90 weight %, or about 95 weight %. In another implementation, the amount of the soap in the cleansing component is greater than 70 weight % and less than 80 weight %. For example, the amount of the soap in the solid cleansing component may be from about 70 weight %, about 71 weight %, about 72 weight %, about 73 weight %, about 74 weight %, or about 75 weight % to about 76 weight %, about 77 weight %, about 78 weight %, about 79 weight %, or about 80 weight %. As discussed above, in a preferred implementation, the cleansing component includes a sodium soap. It should be appreciated, however, that the soap of the cleansing composition may include about 1% to about 25% of any one or more of the ammonium soaps, the potassium soaps, the magnesium soaps, the calcium soaps, or the like, or combinations thereof.

In at least one implementation, the soap may refer to the salts of fatty acids that may typically be used to make soap bars. For example, soap may be or include a mixture or blend of about 60 weight % to about 80 weight % C₁₆-C₁₈ fatty acids and about 20 weight % to about 40 weight % C₁₂-C₁₄ fatty acids, based on the total weight of the soap. The C₁₆-C₁₈ fatty acids may be obtained from tallow and the C₁₂-C₁₄ fatty acids may be obtained from lauric, palm kernel, or coconut oils. In some example, the soap may be or include a mixture or blend of about 60 weight % to about 80 weight % or about 65% to about 75%, or about 70% C16-C18 fatty acids, and about 10 weight % to about 30 weight %, about 15 weight % to about 25 weight %, about 20 weight % to about 40 weight %, about 25% to about 35%, or about 30% C₁₂-C₁₄ fatty acids, based on the total weight of the soap.

The soap of the cleansing component may be or include one or more surfactants. For example, the soap may include one or more anionic surfactants, one or more amphoteric surfactants, one or more cationic surfactants, one or more zwitterionic surfactants, one or more nonionic surfactants, or mixtures thereof. Any other surfactant may also be present in the soap including, but not limited to, sulfate, sulfonate alpha olefin sulfonates, isethionates such as SCI, N-alkyl or N-acyl taurates, sulfosuccinate, phosphates, glycinates, amphoteric surfactants, such as betaines, sulfobetaines or the like, and nonionic surfactants, such as alkanolamide, alkylpolyglycosides.

The solid cleansing composition and the cleansing component or soap thereof may include water. Water of the solid cleansing composition and the cleansing component thereof may be deionized water, demineralized water, and/or softened water. Water of the cleansing component may be separate from the water of other components of the solid cleansing composition. For example, water of the soap may be separate from water in the hydrolyzed protein solution. Water may make up the balance of the solid cleansing composition. For example, the amount of water in the solid cleansing composition may be from about 1 weight % to about 10 weight %, about 10 weight % to about 20 weight %, about 12 weight % to about 18 weight %, or about 14 weight % to about 16 weight %. In another example, the amount of water in the solid cleansing composition may be at least 10 weight %, at least 11 weight %, at least 12 weight %, at least 13 weight %, at least 14 weight %, at least 15 weight %, at least 16 weight %, or at least 17 weight %. In at least one implementation, the amount of water may be about 10 weight %, about 11 weight %, about 12 weight %, about 13 weight %, about 14 weight %, or about 15 weight %. The amount of water in the solid cleansing composition may include free water added and water introduced with other components or materials of the solid cleansing composition. For example, the amount of water in the solid cleansing composition may include free water and water associated with the soap, the hydrolyzed protein solution, and/or any other component of the solid cleansing composition.

The solid cleansing composition may include one or more humectants. Illustrative humectants may include, but are not limited to, one or more of ascorbic acid, ascorbyl dipalmitate, acetamide MEA or acetamide monoethanolamine, glucose glutamate, glucuronic acid, triethanolamine salt of lactic acid (TEA-lactate), pyroglutamic acid triethanolamine salt (TEA-PCA), corn syrup, fructose, glucose, glycerin, glycol, 1,2,6-hexanetriol, sodium lactate, sodium salt of pyroglutamic acid (sodium PCA), hydrogenated starch hydrolysate, inositol, lactic acid, lactose, mannitol, pyroglutamic acid (PCA), PEG-10 propylene glycol, polyamino sugar condensate, propylene glycol, pyridoxine dilaurate, saccharide hydrolysate, hydroxystearyl methylglucamine, glucamine, maltitol, mannitol, methyl gluceth-10, methyl gluceth-20, riboflavin, PEG-4, PEG-6, PEG-8, PEG-9, PEG-10, PEG-12, PEG-14, PEG-16, PEG-18, PEG-20, PEG-32, PEG-40, where PEG are polymers of ethylene glycol, glutamic acid, glycereth-7, glycereth-12, glycereth-26, saccharide isomerate, sorbeth-20, sorbitol, sucrose, thioglycerin, tris-(hydroxymethyl)nitromethane, tromethamine, histidine, PEG-75, PEG-135, PEG-150, PEG-200, PEG-5 pentaerythritol ether, polyglyceryl sorbitol, sorbitol, urea, xylitol, or the like, or combinations thereof. In a preferred implementation, the solid cleansing composition includes glycerin.

In at least one implementation, the solid cleansing composition may include one or more free fatty acids configured to provide enhanced skin feel benefits. For example, the solid cleansing composition may include the fatty acids to provide softer or smoother feeling skin. Illustrative fatty acids may include, but are not limited to, such as palm kernel oil, palm oil, coconut oil, olive oil, laurel oil, or the like, or combinations thereof. Illustrative fatty acids may also include animal fats, such as tallow. Illustrative fatty acids may also include, but are not limited to, fatty acid sources having fatty acid distributions similar or substantially similar to natural or synthetic fatty acid sources (e.g., natural animal fats or oils, natural vegetable fats or oils, individual fatty acids, etc.). The free fatty acids may be separate from the fatty acids associated with the plant oils. In a preferred implementation, the fatty acid sources may be natural fatty acid sources.

The free fatty acids may be present in the solid cleansing composition in an amount of from about 0.5 weight % to about 15 weight %, based on the total weight of the solid cleansing composition. For example, the free fatty acids may be present in an amount of from 0.5 weight %, about 1 weight %, about 1.5 weight %, about 2 weight %, about 3 weight %, about 4 weight %, about 5 weight %, about 6 weight %, or about 7 weight % to about 8 weight %, about 9 weight %, about 10 weight %, about 11 weight %, about 12 weight %, about 13 weight %, about 14 weight %, or about 15 weight %, based on the total weight of the solid cleansing composition. In another example, the free fatty acids may be present in an amount of from about 0.5 weight % to about 15 weight %, about 1 weight % to about 15 weight %, about 1.5 weight % to about 15 weight %, about 5 weight % to about 10 weight %, preferably about 6 weight % to about 9 weight %, or more preferably about 7 weight % to about 8 weight %, based on the total weight of the solid cleansing composition.

The solid cleansing composition may include one or more skin care agents. Any suitable skin care agents that do not adversely affect the stability and/or efficacy of the solid cleansing composition may be used. In at least one implementation, the skin care agent may include an emollient configured to maintain a soft, smooth, and pliable appearance to the skin. As is known by those skilled in the art, the emollients may function by remaining on the surface of the skin or in the stratum corneum to act as a lubricant, to reduce flaking, and/or to improve the appearance of the skin.

The skin care agents may generally include one or more polymers (e.g., polyvinylpyrrolidine), protein derivatives (e.g., derivatized hydrolyzed wheat protein), ethoxylated fatty ethers, cellulosics (e.g., hydroxyethylcellulose), or the like, or mixtures and combinations thereof. Illustrative skin care agents may include, but are not limited to, esters comprising an aliphatic alcohol having about 2 to about 18 carbon atoms condensed with an aliphatic or aromatic carboxylic acid including about 8 to about 20 carbon atoms (e.g., isopropyl myristate, decyl oleate, cetearyl isononanate, etc.). The esters may be straight chained or branched. In a preferred implementation, the ester has a molecular weight of less than about 500.

Other skin care agents may include, but are not limited to, polyvinyl-pyrrolidone, polyquaternium-4, polyquaternium-6, polyquaternium-7, polyquaternium-10, guar gum derivatives, hydroxypropylmethylcellulose, hydroxyethylcellulose, a polyethylene glycol, a methyl ether of a polyethylene glycol, quaternium-79, wheat germamidopropyl hydroxypropyl dimonium hydrolyzed wheat protein, stearyl methicone, dimethicone copolyol, dimethicone propyl PG betaine, poly(sodium styrene sulfonate), sorbitan oleate, steareth-2, steareth-21, isoceteth-20, PEG-7 glyceryl cocoate, PEG-75 lanolin, glycereth-26, PPG-5-ceteth-20, a C₁₂-C₂₀ alcohol, canola oil, glyceryl laurate, triglyceryl monostearate, glyceryl monostearate, vitamin E acetate, sunflower seed amidopropylethyldimonium ethylsulfate, sodium PEG-7 olive oil carboxylate, PPG-1 hydroxyethyl caprylamide, PPG-2 hydroxyethyl cocamide, mineral oil, petrolatum, aloe barbadensis, isostearamidopropylmorpholine lactate, strontium acetate, palmitamidopropyltrimonium chloride, or the like, or combinations thereof. In a preferred implementation, the skin care agent excludes, is free, or substantially free of the polyquats (e.g., polyquaternium-4/6/7/10, etc.).

The solid cleansing composition may include one or more salts capable of or configured to modify the one or more surfactants of the solid cleansing composition. For example, the salts may be configured to at least partially modify a cloud point of the surfactants to thereby control the haze or transparency of the cleansing composition. The salts may be or include one or more inorganic salts including, but not limited to, sodium sulfate, magnesium sulfate, sodium chloride, sodium citrate, or the like, or combinations thereof. The amount of any one or more of the salts may be at least partially determined by the type and/or amount of the surfactants included in the solid cleansing composition. In at least one implementation, the amount of any one or more of the salts may be about 0.1 weight %, 0.2 weight %, 0.3 weight %, 0.4 weight %, or 0.5 weight % to about 0.6 weight %, 0.7 weight %, 0.8 weight %, 0.9 weight %, or about 1.0 weight %.

The solid cleansing composition may include one or more additional optional ingredients. Illustrative optional ingredients may include, but are not limited to, one or more dyes, fragrances (e.g., limonene, ethyl butyrate, linalool, and/or oils, such as citronellol, coumarin, benzyl salicylate, etc.), buffers and buffering agents (e.g., inorganic phosphates, sulfates, and carbonates), pH adjusters (e.g., acids and/or bases), preservatives (e.g., parabens, hydantoins, imidazolines, etc.), thickeners, viscosity modifiers, antioxidants (e.g., etidronic acid, etc.), foam enhancers, chelating agents (e.g., EDTA, phosphates, pentasodium pentetate, etidronic acid, etc.), skin conditioning agents, opacifiers, hydric solvents, hydrotropes, antimicrobials (e.g., trichlorocarbanilide (TCC), triclosan, geraniol, carvacrol, citral, eucalyptol, catechol, 4-allylcatechol, hexyl resorcinol, methyl salicylate, etc.), sunscreen actives, anti-aging compounds, vitamins, essential oils and extracts (e.g., rosewood, jojoba, etc.), polyols, titanium dioxide, abrasives (e.g., particulate matter), acaricidal agents (e.g., benzyl benzoate), or the like, or combinations thereof.

Illustrative antimicrobials may include, but are not limited to, triclocarban, triclosan, or the like, or combinations thereof. Illustrative anti-aging compounds may include, but are not limited to, alpha hydroxy acids, beta hydroxy acids, or the like, or combinations thereof. Illustrative sunscreen actives may include, but are not limited to, butyl methoxy benzoylmethane, or the like, or combinations thereof. Illustrative polyols may include, but are not limited to, glycerol, sorbitol, propylene glycol, polyethylene glycol, or the like, or combinations thereof. Illustrative abrasives or particulate matter may include, but are not limited to, silica, talc, calcium carbonate, polyethylene beads, jojoba beads, lufa, oat flour, or the like, or combinations thereof. Illustrative vitamins may include, but are not limited to, vitamins such as vitamin A, E, K, C, or combinations thereof.

The preservatives may be included in the solid cleansing composition in an amount greater than 0.00 weight % and less than or equal to about 3.0 weight % or less than or equal to about 2.0 weight %. Illustrative preservatives may include, but are not limited to, benzalkonium chloride; benzethonium chloride, 5-bromo-5-nitro-1,3-dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynl butyl carbamate, butyl paraben; ethyl paraben; methyl paraben; propyl paraben, mixture of methyl isothiazolinone/methyl-chloroisothiazoline in a 1:3 wt. ratio; mixture of phenoxythanol/butyl paraben/methyl paraben/propylparaben; 2-phenoxyethanol; tris-hydroxyethyl-hexahydrotriaz-ine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadam-antane chloride; sodium benzoate; organic acids, sorbic acid, lactic acid, citric acid, or the like, or combinations thereof.

In an exemplary implementation, the solid cleaning composition includes a cleansing component, such as a sodium soap including any one or more of sodium palmitate, sodium oleate, sodium laurate, sodium stearate, glycerin, or combinations thereof. The solid cleansing composition further includes water, glycerin, one or more perfumes or fragrances, one or more hydrolyzed proteins, one or more plant seed oils, titanium dioxide, etidronic acid, pentasodium pentetate, limonene, citronellol, coumarin, benzyl benzoate, geraniol, linalool, benzyl salicylate, or any combination thereof.

In at least one implementation, the solid cleansing composition disclosed herein may increase or maintain hydration of skin and/or reduce inflammation/irritation of skin as compared to the same or a similar solid cleansing composition without the hydrolyzed protein (hydrolyzed milk protein) and/or the plant seed oil (flaxseed oil).

In another implementation, the solid cleansing composition disclosed herein may provide one or more attributes comparable to or relatively greater than conventional personal care compositions incorporating PQ-6 or a combination of PA-6 and petrolatum. The one or more attributes may be or include, but are not limited to, lather, skin feel, or combinations thereof. Lather may refer to any one or more of the following: bar glide on hands, flash foam, bubble size, stickiness, foam amount, whiteness of foam, density of foam, peaking, foam thickness on hands, slipperiness of hands (wet), slipperiness of hands (dry), bar glide on arms, foam thickness on arm, time to rinse, or combinations thereof. Skin feel may refer to any one or more of the following: wet slip feel under water, amount of residue when wet, slipperiness after rinsing, tightness, tackiness, slipperiness, smoothness, gloss, white residue after washing, amount of residue after washing, or combinations thereof.

In an exemplary implementation, as summarized in Table 1, the base bar soap composition may include soap chips, glycerin, free fatty acids, water, perfume or fragrances, as well as minors and/or excipients. The soap chip may include sodium tallowate and/or sodium palm kernelate. The soap chip may be present in an amount of about 73.3 weight %, the glycerin may be present in an amount of about 4.5 weight %, the free fatty acids may be present in an amount of about 1.5 weight % or about 7 weight %, the fragrance may be presence in an amount of about 1.5 weight %, and/or the minor/excipients may be present in an amount of about 2 to about 2.0375 weight %, based on the total weight of the base bar soap composition.

In at least one implementation, the bar soap compositions disclosed herein may include the base bar soap composition and flaxseed oil (FSO), hydrolyzed milk protein solution (HMPS), or combinations thereof. For example, the bar soap composition may include the base bar soap composition and FSO, where FSO may be present in an amount of about 0.5 weight %, based on the total weight of the bar soap composition. In another example, the bar soap composition may include the base bar soap composition, FSO, and HMPS, where FSO is present in an amount of about 0.5 weight %, and the HMPS is present in an amount of about 1 weight %, based on the total weight of the bar soap composition.

The present disclosure may provide methods for preparing a personal care composition, such as a solid cleansing composition. The method for preparing the solid cleansing composition may include contacting soap with one or more plant oils and/or one or more hydrolyzed proteins. The one or more plant oils may be or include flaxseed oil. The one or more hydrolyzed proteins may be or include hydrolyzed milk protein. In another example, the one or more plant oils may be or include flaxseed oil, and/or the one or more hydrolyzed proteins may be or include an aqueous solution including hydrolyzed milk protein (e.g., 22% aqueous solution of hydrolyzed milk protein).

The present disclosure may also provide methods for maintaining or increasing hydration of skin. The present disclosure may further provide methods for treating one or more dry skin conditions (e.g., xerosis, psoriasis, dermatitis, eczema, etc.). The method may include contacting surfaces of the skin with any one or more of the solid cleansing composition disclosed herein with the skin of a mammal or human in need thereof.

The present disclosure may also provide methods for deodorizing skin. As used herein, the expression “malodor reduction,” “deodorant ability,” “deodorizing skin,” or the like may refer to the ability of a substance to suppress, neutralize, and/or mask odors of perspiration and/or other body odors. The method may include contacting surfaces of the skin with any one or more of the solid cleansing compositions disclosed herein with the skin of a mammal or human in need thereof.

The present disclosure may further provide a use of a solid cleansing composition including a soap, one or more plant oils, and/or one or more hydrolyzed proteins for deodorizing and/or reducing malodor of skin.

The present disclosure may further provide a use of a solid cleansing composition including a soap, one or more plant oils, and/or one or more hydrolyzed milk proteins for deodorizing and/or reducing malodor of skin.

The present disclosure may further provide a use of a solid cleansing composition including a soap, one or more plant oils, and/or one or more hydrolyzed proteins for maintaining or increasing hydration of skin. The present disclosure may also provide a use of a solid cleansing composition including a soap, one or more plant oils, and/or one or more hydrolyzed proteins for treating one or more dry skin conditions (e.g., xerosis, psoriasis, dermatitis, eczema etc.). The present disclosure may also provide a use of a solid cleansing composition including a soap, one or more plant oils, and/or one or more hydrolyzed proteins for treating, inhibiting, or preventing irritation or inflammation of skin.

The present disclosure may further provide a use of a solid cleansing composition including a soap, one or more plant oils, and/or one or more hydrolyzed milk proteins for maintaining or increasing hydration of skin. The present disclosure may also provide a use of a solid cleansing composition including a soap, one or more plant oils, and/or one or more hydrolyzed proteins for treating one or more dry skin conditions (e.g., xerosis, psoriasis, dermatitis, eczema etc.). The present disclosure may also provide a use of a solid cleansing composition including a soap, one or more plant oils, and/or one or more hydrolyzed milk proteins for treating, inhibiting, or preventing irritation or inflammation of skin.

The present disclosure may further provide a method for replacing one or more synthetic or non-natural components of a personal care composition, such as a solid cleansing composition. For example, the present disclosure may provide methods for replacing polyquats, such as PQ-6, or a combination of polyquats and petrolatum with a plant oil, such as flaxseed oil or a plant oil source.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

A base bar soap composition (1) was prepared by combining the ingredients/components according to Table 1. Four bar soap compositions (2)-(5) were then prepared by adding varying amounts of petrolatum, Polyquaternium 6 (PQ6), flaxseed oil (FSO), aloe, and/or hydrolyzed milk protein solution (HMPS) (MILK TEIN NPNF®; 22% hydrolyzed milk protein in water) to the base bar soap composition (1), according to Table 2. The hydrolyzed milk protein solution (MILK TEIN NPNF®) was obtained from Tri-K Industries, Inc. of Denville, NJ.

TABLE 1
Composition of Base Bar Soap Composition (1)

| | Amount |
|---|---|
| Ingredient/Component | (wt %) |
| Soap chips containing | 73.3 |
| sodium tallowate and | |
| sodium palm kernelate | |
| Glycerin | 4.5 |
| Free Fatty Acids | 7 |
| Demineralized water | Q.S. |
| Perfume | 1.5 |
| Minors/Excipients | 2-2.0375 |

TABLE 2
Compositions of Control and Test Bar Soap Compositions (1)-(5)

| | Base Bar Soap | | | | |
|---|---|---|---|---|---|
| | Composition | FSO | HMPS | PQ6 | Petrolatum |
| # | (wt %) | (wt %) | (wt %) | (wt %) | (wt %) |
| (1) | 100.0 | — | — | — | — |
| (2) | 99.5 | 0.5 | — | — | — |
| (3) | 95.75 | — | — | 0.71 | 3.5 |
| (4) | 98.46 | 0.5 | 1 | — | — |
| (5) | 95.25 | 0.5 | — | 0.71 | 3.5 |

### Example 2

Each of control (1) and test (2)-(5) bar soap compositions were evaluated for lather and skin feel. Attributes of lather that were evaluated included: flash foam at 5 min and 10 min, bubble size, stickiness, foam amount, density of foam, foam thickness (hands and arms), bar glide (hands and arms), and whiteness of foam. Attributes of skin feel that were evaluated included: slipperiness (time=0 min and 5 min), smoothness (time=0 min, 5 min, and 10 min), and amount of residue (time=5 min, 10 min), gloss (time=5 min, 10 min).

A nine member expert panel according to the Bar Soap Expert Panel Standard Procedure was utilized to evaluate each of the bar soap compositions (1)-(5). Each of the bar soap compositions (1)-(5) was evaluated over a two day period. Each day, two of the bar soap compositions (1)-(5) were evaluated by the expert panel in a randomized and balanced form. Each of the expert panel evaluated foam and skin feel attributes, during hand and arm washing using a 10-point scale from 0 to 10, where 0 was the lowest score denoting poor foam and skin feel attributes. Statistical significance was determined at a 90% confidence level via two-tail analysis, and the Tukey method was utilized for grouping and detecting significant differences. Evaluations were performed at varying time points, namely, immediately after washing (time=0 min), 5 min, 10 min, and 20 min after washing. The results of the evaluation are summarized in Tables 3-6. Specifically, Table 3 summarizes the results comparing lather attributes of the bar soap composition (2) with FSO against the bar soap composition (1) without FSO as the standard. Table 4 summarizes the results comparing skin feel attributes of the bar soap composition (2) with FSO against the bar soap composition (1) without FSO as the standard. Table 5 summarizes the results comparing lather attributes of the bar soap composition (3) with bar soap compositions (4) and (5), where (3) was used as the standard. Table 6 summarizes the results comparing skin feel attributes of the bar soap composition (3) against bar soap compositions (4) and (5), where (3) was used as the standard. As discussed above, the 10-point scale from 0 to 10 were utilized and analyzed with statistical significance. As indicated in Tables 3-6, the results are tabulated as parity (=), lower score (−), or higher score (+) based on the scaled score.

TABLE 3
Comparison of Lather of (2) Base + FSO Against (1) Base

(2)

ATTRIBUTE (HANDS)

| | |
|---|---|
| Bar Glide on Hands | − |
| Flash Foam (Time = 5 min) | + |
| Bubble Size | + |
| Flash Foam (Time = 10 min) | + |
| Stickiness | + |
| Foam Amount | + |
| Whiteness of Foam | − |
| Density of Foam | + |
| Peaking | = |
| Foam Thickness | + |
| Slipperiness of hands (wet) | − |
| Slipperiness of hands (dry) | − |

ATTRIBUTE (ARMS)

| | |
|---|---|
| Bar Glide on Arms | − |
| Foam Thickness | + |
| Time to Rinse | = |

‘−’ means lower score
‘+’ means higher score
‘=’ means parity

TABLE 4
Comparison of Skin Feel of (2) Base + FSO Against (1) Base

(2)

SKIN FEEL AFTER 4^{TH} WASH

| | |
|---|---|
| Wet Slip under water (T = 5 min) | = |
| Wet Slip under water (T = 10 min) | = |
| Wet Slip under water (T = 15 min) | = |
| Amount of Residue when wet | = |
| Slipperiness after rinsing | = |

TIGHTNESS

| | |
|---|---|
| Tightness (T = 0 min) | = |
| Tightness (T = 5 min) | = |
| Tightness (T = 10 min) | = |
| Tightness (T = 20 min) | = |

TACKINESS

| | |
|---|---|
| Tackiness (T = 0 min) | + |
| Tackiness (T = 5 min) | = |
| Tackiness (T = 10 min) | = |
| Tackiness (T = 20 min) | = |

SLIPPERINESS

| | |
|---|---|
| Slipperiness (T = 0 min) | + |
| Slipperiness (T = 5 min) | + |
| Slipperiness (T = 10 min) | = |
| Slipperiness (T = 20 min) | = |

SMOOTHNESS

| | |
|---|---|
| Smoothness (T = 0 min) | + |
| Smoothness (T = 5 min) | + |
| Smoothness (T = 10 min) | + |
| Smoothness (T = 20 min) | − |

GLOSS

| | |
|---|---|
| Gloss (T = 0 min) | = |
| Gloss (T = 5 min) | − |
| Gloss (T = 10 min) | = |
| Gloss (T = 20 min) | − |

WHITE RESIDUE

| | |
|---|---|
| White Residue (T = 5 min) | = |
| White Residue (T = 10 min) | = |
| White Residue (T = 20 min) | + |

AMOUNT OF RESIDUE

| | |
|---|---|
| Amount of Residue (T = 0 min) | = |
| Amount of Residue (T = 5 min) | + |
| Amount of Residue (T = 10 min) | + |
| Amount of Residue (T = 20 min) | = |

‘−’ means lower score
‘+’ means higher score
‘=’ means parity

As indicated in Table 3, the bar soap composition (2) was superior to the base bar soap composition without FSO in the majority of lather attributes. Further, Table 4 illustrated that the bar soap composition (2) having FSO was at least parity with respect to a majority of skin feel attributes, and further demonstrated greater scores relative to the base soap composition (1) with respect to smoothness and amount of residue.

TABLE 5
Comparison of Lather between the bar soap
composition (4) and (5) against (3)

| | |
|---|---|
| (4) | (5) |

ATTRIBUTE (HANDS)

| | | |
|---|---|---|
| Bar Glide on Hands | + | + |
| Flash Foam (Time = 5 min) | + | + |
| Bubble Size | + | + |
| Flash Foam (Time = 10 min) | = | + |
| Stickiness | + | = |
| Foam Amount | = | + |
| Whiteness of Foam | − | + |
| Density of Foam | = | + |
| Peaking | = | + |
| Foam Thickness | = | + |
| Slipperiness of hands (wet) | + | = |
| Slipperiness of hands (dry) | + | = |

ATTRIBUTE (ARMS)

| | | |
|---|---|---|
| Bar Glide on Arms | = | = |
| Foam Thickness | − | + |
| Time to Rinse | = | + |

‘−’ means lower score
‘+’ means higher score
‘=’ means parity

TABLE 6
Comparison of Skin Feel between the bar
soap composition(4) and (5) against (3)

| | |
|---|---|
| (4) | (5) |

SKIN FEEL AFTER 4^{TH} WASH

| | | |
|---|---|---|
| Wet Slip under water (T = 5 min) | = | + |
| Wet Slip under water (T = 10 min) | + | + |
| Wet Slip under water (T = 15 min) | + | + |
| Amount of Residue when wet | = | = |
| Slipperiness after rinsing | + | + |

TIGHTNESS

| | | |
|---|---|---|
| Tightness (T = 0 min) | = | = |
| Tightness (T = 5 min) | = | = |
| Tightness (T = 10 min) | = | − |
| Tightness (T = 20 min) | = | − |

TACKINESS

| | | |
|---|---|---|
| Tackiness (T = 0 min) | − | − |
| Tackiness (T = 5 min) | = | = |
| Tackiness (T = 10 min) | = | = |
| Tackiness (T = 20 min) | = | = |

SLIPPERINESS

| | | |
|---|---|---|
| Slipperiness (T = 0 min) | + | + |
| Slipperiness (T = 5 min) | + | + |
| Slipperiness (T = 10 min) | + | + |
| Slipperiness (T = 20 min) | = | + |

SMOOTHNESS

| | | |
|---|---|---|
| Smoothness (T = 0 min) | + | + |
| Smoothness (T = 5 min) | + | + |
| Smoothness (T = 10 min) | + | + |
| Smoothness (T = 20 min) | + | + |

GLOSS

| | | |
|---|---|---|
| Gloss (T = 0 min) | = | = |
| Gloss (T = 5 min) | = | = |
| Gloss (T = 10 min) | = | = |
| Gloss (T = 20 min) | = | = |

WHITE RESIDUE

White Residue (T = 5 min)
White Residue (T = 10 min)
White Residue (T = 20 min)

AMOUNT OF RESIDUE

| | | |
|---|---|---|
| Amount of Residue (T = 0 min) | = | = |
| Amount of Residue (T = 5 min) | + | = |
| Amount of Residue (T = 10 min) | + | = |
| Amount of Residue (T = 20 min) | + | = |

‘−’ means lower score
‘+’ means higher score
‘=’ means parity

In comparing the bar soap composition (3) with the bar soap compositions (4) and (5) in Tables 5 and 6, it was demonstrated that the bar soap composition (4) without the combination of petrolatum and PQ6 was at least parity and in some cases superior or had relatively greater scores with respect to many of the lather attributes except foam thickness. The bar soap composition (4) also exhibited superior skin feel with respect to smoothness and slipperiness. As further illustrated in Tables 5 and 6, the bar soap composition (5) exhibited superior lather attributes, and also demonstrated superior skin feel attributes with respect to slipperiness and smoothness, as well as wet slip under water.

### Example 3

The bar soap compositions (1) and (2) of Example 1 were evaluated for inflammation and irritation. Specifically, an in vitro study was conducted on keratinocyte cell skin models to observe the production of Interleukin 1 alpha (IL-1α) in the absence and presence of FSO. It should be appreciated that IL-1α, which is also known as hematopoietin 1, is a cytokine of the interleukin 1 family that is encoded by the IL1α gene. It should further be appreciated that interleukin 1 is responsible for the production of inflammation and irritation, and is widely accepted as a biomarker to evaluate skin irritation.

To evaluate the IL-1α production, the keratinocyte cell skin models were grown to at least 60% confluency and placed in respective wells of a well plate. 3D human skin models, obtained from MatTek Corp. of Ashland, MA, were utilized as the models in the in vitro study, and the IL-1α production was monitored with an Elisa Kit. To conduct the in vitro study, each of bar soap compositions (1) and (2) was diluted in distilled water in a ratio of 1:20, and subsequently further diluted in phosphate-buffered saline (PBS) in a ratio of 1:5 to thereby produce a solution having 1 weight % of the respective bar soap compositions (1) and (2).

To evaluate the production of IL-1α, each of the 1 weight % bar soap compositions (1) and (2) was contacted with the 3D skin models. Particularly, 30 μL of the bar soap composition (1) or (2) was added and evenly spread along a top surface of the 3D skin model and incubated at 37° C. for one hour. The 3D skin model was then washed with PBS until the respective bar soap composition (1) or (2) was completely removed (about 5-8 washes). The 3D skin model was then transferred to a dish containing new media and further incubated at 37° C. for 24 hours. The tissue was collected with HSP lysis buffer, lysed twice at 15.01/s for about 15 min and frozen at about −80° C. The media was then collected for the Elisa assay.

TABLE 7
Amount of IL-1α Measured in Bar Soap Compositions (1) and (2)

| # | Sample | Amount of IL-1α | Change from Control |
|---|---|---|---|
| 1 | Base Soap | 59.2 | — |
| 2 | Base Soap + | 43.7 | 15.5 |
| | 0.5 wt % FSO | | |

As illustrated in Table 7, the inclusion of FSO significantly decreased the amount of IL-1α measured. As such, it was demonstrated that the inclusion of FSO reduces skin irritation.

### Example 4

The bar soap compositions (1) and (2) prepared in Example 1 were evaluated in vitro on skin tissue models to observe the production of natural moisturizing factors (NMFs), particularly, Caspase-14. Each of bar soap compositions (1) and (2) was tested as 1% soap samples, similar to Example 3. Particularly, each of the bar soap compositions (1) and (2) was diluted in water in a weight ratio of 1:20, and subsequently diluted in phosphate-buffered saline (PBS) in a weight ratio of 1:5. 3D human skin models obtained from MatTek Corp. of Ashland, MA, were utilized as the models in the in vitro study, and the Caspase-14 production was monitored with an ELISA Kit.

To conduct the in vitro study, 30 um of respective 1% solutions of the bar soap compositions (1) or (2) were topically applied to respective 3D human skin models and incubated at about 37° C. for about 1 hour. After about 1 hour, each of the 3D human skin models was thoroughly and gently washed with PBS about 5 to about 8 times. Each of the 3D human skin models was then placed in fresh media and incubated at about 37°° C. for about 24 hours. The 3D human skin models were then collected and lysed four times with lysis buffer at 15.01/s for 15 minutes. After lysing with the lysis buffer, each of the lysed samples was frozen or maintained at about −80° C. The production of NMF biomarker Caspase-14, as measured by the ELISA kit, from respective human skin models treated with each of the bar soap compositions (1) and (2) is summarized in Table 8. All measurements were done in triplicate and averaged unless indicated otherwise. All measurements were normalized to the total protein in each of the 3D human skin models.

TABLE 8
Amount of Filaggrin Measured from Skin Models
Treated with 2% Solutions of Control and Test
Liquid Cleansing Compositions (1) and (2)

| | Amount | | | |
|---|---|---|---|---|
| | of FSO | Amount of | | Change |
| # | (wt %) | filaggrin | Std Dev | from Control |
| (1) | 0 | 0.109 | 0.05 | — |
| (2) | 0.5 | 0.168 | 0.017 | 0.059 |

As illustrated in Table 8, the presence of FSO increased the amount of Caspase-14. It should be appreciated that the increased amount of biomarker Caspase-14 indicates the production of NMF, as Caspase-14 is utilized in the degradation of filaggrin to produce NMF in skin.

### Example 5

A base bar soap composition (6) was prepared by combining the ingredients/components according to Table 9. Six bar soap compositions (7)-(12) were then prepared by adding varying amounts of flaxseed oil (FSO), aloe, and HMPS to the base bar soap composition (6), according to Table 10.

TABLE 9
Composition of Base Bar Soap Composition (6)

| | Amount |
|---|---|
| Ingredient/Component | (wt %) |
| Soap chips containing | 69.1 |
| sodium tallowate and | |
| sodium palm kernelate | |
| Glycerin | 9.5 |
| Free Fatty Acids | 1.5 |
| Demineralized water | Q.S. |
| Minors/Excipients | 2-2.0375 |

TABLE 10
Compositions of Control and Test Bar Soap Compositions (7)-(12)

| | Base Bar Soap | FSO | Aloe | HMPS |
|---|---|---|---|---|
| # | Composition (wt %) | (wt %) | (wt %) | (wt %) |
| (7) | 100% | — | — | — |
| (8) | 99.5 | 0.5 | — | — |
| (9) | 99.26% | 0.5 | 0.0375 | 0.2 |
| (10) | 99.96% | — | 0.0375 | — |
| (11) | 99.3 | 0.5 | — | 0.2 |
| (12) | 99.8 | — | — | 0.2 |

### Example 6

Each of the bar soap compositions (7)-(12) were evaluated for deodorant ability or their respective ability to deodorize. As used herein, the expression “deodorant ability” or the like refers to the ability of a substance to suppress, neutralize, and/or mask odor of perspiration and/or other body odors.

In order to evaluate the deodorant ability, 216 adult male volunteers aged 18-55 were enrolled and 183 completed the study. The subjects participated in a conditioning period during which time they refrained from using antiperspirants and all antibacterial products for three weeks and all deodorant products for 14 days. During this time they all washed and bathed with a control, standardized bar soap. The study was double-blind, balanced, complete block design. Bar soap compositions (7) and (8) were paired, bar soap compositions (9) and (10) were paired, and bar soap compositions (11) and (12) were paired. Each of the pairs were tested on one subject's axilla, one on each axilla. For example, a single subject would be tested with bar soap composition (7) on one axilla and bar soap composition (8) on the other axilla.

On Day 1 (AM), each of the subjects participated in a supervised wash, washing both underarms using the control, standardized bar soap. About 8 hours later (Day 1, PM), subjects returned for baseline odor rating by a four (4) member expert panel. Both axillae were evaluated or sniffed using the 0-10 odor intensity scale where 0 indicates no malodor, 5 indicates moderate malodor, and 10 indicates extremely strong malodor. Subjects with a mean odor score of 5 or greater were qualified to participate in the test phase.

On Day 2 (AM) qualified subjects from Day 1 returned for a supervised wash using the respective bar soap pairs as discussed above. Test products were randomly assigned to the right and left axillae. These washes were repeated for two days for a total of three washes. The wash procedure for the bar soap was as follows: washing was conducted with running tap water maintained at 90-95° F.; each subject wore disposable gloves to avoid cross contamination; subjects wetted their right axilla and the respective bar soap composition; then the bar soap composition was applied directly to the right axilla and washed for 30 seconds(s); the bar soap composition was then applied to a wet, disposable washcloth and lathered for 10 s and then the washcloth was applied to the right axilla for an additional 30 s; the right axilla was then thoroughly rinsed and patted dry with a towel; the left axilla was then washed with the same procedure. After about 15 min, each of the subjects received a white cotton t-shirt to wear until the next visit. On Day 4 (PM) subjects returned for a 5 hour (h), 8h, and 12h odor rating.

Data was analyzed using a general linear model to test for treatment differences while controlling for panelist and axilla. The data was analyzed using ANOVA. The results are summarized in Table 11.

TABLE 11
Compositions of Control and Test Bar Soap Compositions (7)-(12)

| | Baseline | 5 h | 5 h | 8 h | 8 h | 12 h | 12 h |
|---|---|---|---|---|---|---|---|
| | Score | Score | Reduction | Score | Reduction | Score | Reduction |
| # | (mean) | (mean) | (%) | (mean) | (%) | (mean) | (%) |
| (7) | 6.34 | 1.80^{a} ± 0.82 | 71.61 | 1.89^{a} ± 0.84 | 70.19 | 2.88^{a} ± 0.93 | 54.57 |
| (8) | 6.34 | 2.68^{b} ± 0.93 | 57.73 | 2.79^{b} ± 0.96 | 55.99 | 3.88^{b} ± 0.93 | 38.80 |
| (9) | 6.27 | 1.28^{c} ± 0.45 | 79.59 | 1.43^{c} ± 0.50 | 77.19 | 2.39^{c} ± 0.55 | 61.88 |
| (10) | 6.27 | 2.23^{d} ± 0.52 | 64.43 | 2.38^{d} ± 0.58 | 62.04 | 3.39^{d} ± 0.56 | 45.93 |
| (11) | 6.23 | 1.25^{e} ± 0.43 | 79.94 | 1.27^{e} ± 0.44 | 79.61 | 2.33^{e} ± 0.47 | 62.60 |
| (12) | 6.23 | 2.25^{f} ± 0.43 | 63.88 | 2.27^{f} ± 0.44 | 63.56 | 3.33^{f} ± 0.47 | 46.55 |

*Means with similar superscripts were not statistically significant from each other (p > 0.05).

As illustrated in Table 11, bar soap composition (8), which included FSO, was significantly different from bar soap composition (7), which did not include FSO at all time points. While both bar soap compositions (7) and (8) exhibited a significant reduction in malodor, the increase in deodorant ability for bar soap composition (8) was significantly greater.

As illustrated in Table 11, bar soap composition (9), which included FSO, was significantly different from bar soap composition (10), which did not include FSO at all time points. While both bar soap compositions (9) and (10) exhibited a significant reduction in malodor, the increase in deodorant ability for bar soap composition (9) was significantly greater.

As illustrated in Table 11, bar soap composition (11), which included FSO, was significantly different from bar soap composition (12), which did not include FSO at all time points. While both bar soap compositions (11) and (12) exhibited a significant reduction in malodor, the increase in deodorant ability for bar soap composition (11) was significantly greater.

As illustrated in Tables 10 and 11, a method of ameliorating a negative effect of aloe on odor reduction of a cleansing composition is also provided. The method includes
(a) identifying a first odor reduction of aloe on the cleansing composition, comprising: (i) providing a first composition comprising a soap, for example composition (7), wherein the first composition has a first odor reduction percentage; and (ii) adding aloe to the first composition to produce a second composition, for example composition (10), having a second odor reduction percentage; wherein the difference (or the normalized difference) between the first odor reduction percentage and the second odor reduction percentage is the first odor reduction of aloe on the cleansing composition;
(b) identifying a second odor reduction of aloe on the cleansing composition, comprising: (iii) providing a third composition, for example composition (11), which comprises a plant oil, a hydrolyzed protein, and the first composition, wherein the third composition has a third odor reduction percentage; (iv) adding aloe to the third composition, to produce a fourth composition, for example composition (9), having a fourth odor reduction percentage; wherein the difference (or the normalized difference) between the third odor reduction percentage and the fourth odor reduction percentage is the second odor reduction of aloe on the cleansing composition; and
(c) applying the fourth composition to a skin for reducing odor of the skin.

Advantageously, the second odor reduction of aloe on the cleansing composition is less than the first odor reduction of aloe on the cleansing composition, indicating the negative effect of aloe is ameliorated in the presence of the plant oil and the hydrolyzed protein.

For example, the first odor reduction of aloe on the cleansing composition may be defined as X=M1−M2 (or normalized X₀=(M1−M2)/M1). M1 stands for the first odor reduction percentage and M2 stands for the second odor reduction percentage. Similarly, the second odor reduction of aloe on the cleansing composition may be defined as Y=M3−M4 (or normalized Y₀=(M3−M4)/M3). M3 stands for the third odor reduction percentage and M4 stands for the fourth odor reduction percentage. It has been observed from Tables 10 and 11 that Y<X and Y₀<X₀.

As illustrated in Tables 10 and 11, compositions (7), (10), (11) and (9) are representative examples of the first, second, third and fourth compositions, respectively. It has been observed from Tables 10 and 11 that X≥7.18% and Y≤2.42%; and X₀≥10% and Y₀≤3%.

As illustrated in Tables 10 and 11, at the 5-hour odor rating, M1=71.61, M2=64.43, M4=79.59, and M3=79.94. As such, X=M1−M2=71.61%−64.43%=7.18%, and X₀=(M1−M2)/M1=(71.61%−64.43%)/71.61%=10%. Y=M3−M4=(79.94%−79.59%=0.35%, and Y₀=(M3−M4)/M3=(79.94−79.59)/79.94=0.44%. Apparently, Y is far less than X, and Y₀ is far less than X₀, indicating the negative effect of aloe is ameliorated in the presence of FSO and HMPS.

At the 8-hour odor rating, we see that M2=62.04, M1=70.19, M4=77.19, and M3=79.61. As such, X=M1−M2=70.19%−62.04%=8.15%, X₀=(M1−M2)/M1=(70.19%−62.04%)/70.19%=11.6%, Y=M3−M4=79.61%− 77.19%=2.42%, and Y₀=(M3−M4)/M3=(79.61%−77.19%)/79.61%=3.0%. Again, Y is far less than X, and Y₀ is far less than X₀, indicating the negative effect of aloe is ameliorated in the presence of FSO and HMPS.

At the 12-hour odor rating, M2=45.93, M1=54.57, M4=61.88, and M3=62.60. As such, X=M1−M2=54.57%−45.93%=8.64%, X₀=(M1−M2)/M1=(54.57%−45.93%)/54.57%=15.8%, Y=M3−M4=62.60%−61.88%=0.72%, and Y₀=(M3−M4)/M3=(62.60%−61.88%)/62.60%=1.2%. Again, Y is far less than X, and Y₀ is far less than X₀, indicating the negative effect of aloe is ameliorated in the presence of FSO and HMPS.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

What is claimed is:

1. A method of ameliorating a negative effect of aloe on odor reduction of a cleansing composition, the method comprising:
(a) identifying a first odor reduction of aloe on the cleansing composition, comprising:
(i) providing a first composition comprising a soap, wherein the first composition has a first odor reduction percentage; and
(ii) adding aloe to the first composition to produce a second composition having a second odor reduction percentage;
wherein the difference (or the normalized difference) between the first odor reduction percentage and the second odor reduction percentage is the first odor reduction of aloe on the cleansing composition;
(b) identifying a second odor reduction of aloe on the cleansing composition, comprising:
(iii) providing a third composition comprising a plant oil, a hydrolyzed protein, and the first composition, wherein the third composition has a third odor reduction percentage;
(iv) adding aloe to the third composition, to produce a fourth composition having a fourth odor reduction percentage;
wherein the difference (or the normalized difference) between the third odor reduction percentage and the fourth odor reduction percentage is the second odor reduction of aloe on the cleansing composition; and
(c) applying the fourth composition to a skin for reducing odor of the skin; and
wherein said second odor reduction of aloe on the cleansing composition is less than said first odor reduction of aloe on the cleansing composition, indicating the negative effect of aloe is ameliorated in the presence of the plant oil and the hydrolyzed protein.

2. The method according to claim 1, wherein said first odor reduction of aloe on the cleansing composition is X=M1−M2 (or normalized X₀=(M1−M2)/M1), in which M1 stands for the first odor reduction percentage and M2 stands for the second odor reduction percentage;
wherein said second odor reduction of aloe on the cleansing composition is Y=M3−M4 (or normalized Y₀=(M3−M4)/M3), in which M3 stands for the third odor reduction percentage and M4 stands for the fourth odor reduction percentage; and
wherein Y<X and Y₀<X₀.

3. The method according to claim 2, wherein X≥7.18% and Y≤2.42%; and wherein X₀≥10% and Y₀≤3%.

4. The method according to claim 2, wherein the plant oil is flaxseed oil (FSO), wherein the hydrolyzed protein is hydrolyzed milk protein solution (HMPS), M2=64.43, M1=71.61, M4=79.59, and M3=79.94 at a 5-hour odor rating,
wherein X=M1−M2=71.61%−64.43%=7.18%,
wherein X₀=(M1−M2)/M1=(71.61%− 64.43%)/71.61%=10%,
wherein Y=M3−M4=(79.94%−79.59%=0.35%,
wherein Y₀=(M3−M4)/M3=(79.94−79.59)/79.94=0.44%, and
wherein Y is far less than X, and Y₀ is far less than X₀.

5. The method according to claim 2, wherein the plant oil is flaxseed oil (FSO), wherein the hydrolyzed protein is hydrolyzed milk protein solution (HMPS), M2=62.04, M1=70.19, M4=77.19, and M3=79.61 at an 8-hour odor rating,
wherein X=M1−M2=70.19%−62.04%=8.15%,
wherein X₀=(M1−M2)/M1=(70.19%−62.04%)/70.19%=11.6%,
wherein Y=M3−M4=79.61%−77.19%=2.42%,
wherein Y₀=(M3−M4)/M3=(79.61%−77.19%)/79.61%=3.0%, and
wherein Y is far less than X, and Y₀ is far less than X₀.

6. The method according to claim 2, wherein the plant oil is flaxseed oil (FSO), wherein the hydrolyzed protein is hydrolyzed milk protein solution (HMPS), M2=45.93, M1=54.57, M4=61.88, and M3=62.60 at a 12-hour odor rating,
wherein X=M1−M2=54.57%−45.93%=8.64%,
wherein X₀=(M1−M2)/M1=(54.57%− 45.93%)/54.57%=15.8%,
wherein Y=M3−M4=62.60%−61.88%=0.72%,
wherein Y₀=(M3−M4)/M3=(62.60%−61.88%)/62.60%=1.2%, and
wherein Y is far less than X, and Y₀ is far less than X₀.

7. The method according to claim 1, wherein the plant oil comprises flaxseed oil.

8. The method according to claim 1, wherein the plant oil is present in an amount of from greater than 0 weight % to less than or equal to 5 weight %, optionally, in an amount of from greater than 0 weight % to less than or equal to 1 weight %, further optionally, in an amount of about 0.5 weight %, based on a total weight of the fourth composition.

9. The method according to claim 1, wherein the hydrolyzed protein comprises hydrolyzed milk protein.

10. The method according to claim 1, wherein the hydrolyzed protein is present in an amount of from greater than 0 weight % to about 1 weight %, optionally, from about 0.15 weight % to about 0.3 weight %, further optionally, from about 0.18 weight % to about 0.22 weight %, further optionally, in an amount of about 0.2 weight %, based on a total weight of the fourth composition.

11. The method according to claim 1, wherein a weight ratio of the plant oil to the hydrolyzed protein is about 2.0:1 to about 3.0:1, optionally, about 2.2:1 to about 2.8:1, about 2.4:1 to about 2.6:1, or about 2.5:1.

12. The method according to claim 1, wherein the hydrolyzed protein is provided by an aqueous hydrolyzed protein solution comprising hydrolyzed milk protein and water, optionally, the hydrolyzed milk protein is present in an amount of about 18 weight % to about 25 weight %, based on a total weight of the aqueous hydrolyzed protein solution.

13. The method according to claim 1, wherein the fourth composition is free of polyquaternium-6 and/or petrolatum.

14. The method according to claim 1, wherein the soap comprise C₁₆-C₁₈ fatty acids and C₁₂-C₁₄ fatty acids, optionally, wherein the C₁₆-C₁₈ fatty acids are present in an amount of from about 60 weight % to about 80 weight %, and wherein the C₁₂-C₁₄ fatty acids are present in an amount of from about 20 weight % to about 40 weight %, based on the total weight of the fourth composition.

15. The method according to claim 1, wherein the soap comprises sodium soap, optionally the soap further comprises one or more of an ammonium soap, a potassium soap, a magnesium soap, a calcium soap, or a combination thereof.

16. The method according to claim 1, wherein the soap comprises alkali metal salts of aliphatic acids having 8 to 22 carbon atoms.

17. The method according to claim 1, wherein the fourth composition further comprises one or more humectants selected from ascorbic acid, ascorbyl dipalmitate, acetamide MEA, glucose glutamate, glucuronic acid, TEA-lactate, TEA-PCA, corn syrup, fructose, glucose, glycerin, glycol, 1,2,6-hexanetriol, sodium lactate, sodium PCA, hydrogenated starch hydrolysate, inositol, lactic acid, lactose, mannitol, PCA, PEG-10 propylene glycol, polyamino sugar condensate, propylene glycol, pyridoxine dilaurate, saccharide hydrolysate, hydroxystearyl methylglucamine, glucamine, maltitol, mannitol, methyl gluceth-10, methyl gluceth-20, riboflavin, PEG-4, PEG-6, PEG-8, PEG-9, PEG-10, PEG-12, PEG-14, PEG-16, PEG-18, PEG-20, PEG-32, PEG-40, glutamic acid, glycereth-7, glycereth-12, glycereth-26, saccharide isomerate, sorbeth-20, sorbitol, sucrose, thioglycerin, tris-(hydroxymethyl)nitromethane, tromethamine, histidine, PEG-75, PEG-135, PEG-150, PEG-200, PEG-5 pentaerythritol ether, polyglyceryl sorbitol, sorbitol, urea, xylitol, or a combination thereof.

18. The method according to claim 17, wherein the humectants comprise glycerin.

19. The method according to claim 1, wherein the fourth composition further comprises one or more free fatty acids, optionally, wherein the one or more free fatty acids comprise one or more of palm kernel oil, palm oil, coconut oil, olive oil, laurel oil, or combinations thereof, and optionally, the one or more free fatty acids are present in an amount greater than 0 weight % to about 15 weight %, based on a total weight of the fourth composition.

20. The method according to claim 1, wherein the fourth composition is a bar soap.
